(19) United States
(12) Patent Application Publication
(10) Pub. No.: US 2025/0073250 A1
(43) Pub. Date: Mar. 6, 2025

(54) Improved synthesis of lysine acetylsalicylate - Glycine particles

(71) Applicant: AspiAir GmbH, Gemünden (DE)

(72) Inventors: Karlheinz Nocker, Gemünden (DE); Ralf Zuhse, Gemünden (DE); Thomas von Schrader, Gemünden (DE); Christian Braune, Gemünden (DE)

(21) Appl. No.: 18/726,412

(22) PCT Filed: Jan. 5, 2023
(86) PCT No.: PCT/EP2023/050175

§ 371 Date: Jul. 3, 2024

(30) Foreign Application Priority Data
Jan. 5, 2022 (EP) 22150312.1

(51) Int. Cl. A61K 31/616; C07C 67/317; C07C 69/92; C07C 227/18; C07C 229/08; C07C 229/26

(52) U.S. Cl. CPC A61K 31/616; C07C 67/317; C07C 69/92; C07C 227/18; C07C 229/08; C07C 229/26; C07B 2200/13

(57) ABSTRACT

The invention provides an improved method for preparing lysine acetylsalicylate⋅glycine (LASAG) that allows for high yields without the need for an addition of seed crystal, as well as yielding controlled, small particle sizes (median particles size preferably <40 μm) and high stability of the LASAG. Advantageously, the method can be performed at room temperature without negatively impacting yield or particle properties. The invention also provides the LASAG obtained from said method, and its uses as a medicine.

### BACKGROUND OF THE INVENTION

Acetylsalicylic acid (or ‘ASA’ for short) has been used in therapy for over 100 years; most commonly known under its trade name Aspirin®. In particular, o-acetylsalicylic acid is widely used as analgesic, antipyretic or antirheumatic agent, as well as a non-steroid anti-inflammatory agent in arthritis, neuralgia or myalgia. Unfortunately, acetylsalicylic acid has a limited solubility in water, which limits its resorption speed and with that the potential application forms. It was found that some acetylsalicylic acid salts show a significantly improved resorption speed. In particular, salts of acetylsalicylic acid with basic amino acids, especially lysine, show a highly-improved resorption speed.

The commonly used salt of acetyl salicylic acid in this context is acetylsalicylic acid lysinate; also referred to as lysine acetylsalicylate, or ‘LASA’ for short. The salt has been known for over 60 years, and has been utilized in several pharmaceutical compositions and applications. An advantage of acetylsalicylic acid lysinate is its high tolerance in oral applications, as well as an increased absorption speed compared to acetylsalicylic acid alone.

A further important compound is lysine acetylsalicylate glycine (depending on the lysine stereoisomer used, sometimes also referred to as L-, D-, or D,L-lysine acetylsalicylate⋅glycine, or L-, D-, or D,L-lysine acetylsalicylate+glycine, or ‘LASAG’ for short), in which the LASA is supplemented with, or associated with, the further amino acid glycine, offering inter alia improved stability properties. The glycine can be added to the LASA either ‘externally’ (e.g., in the form of a solid mixture with the LASA powder, as described in WO2018115434A1), or ‘internally’ in the form of a co-crystal (i.e., when the glycine is added already during the LASA crystallization step so that the glycine is incorporated within the LASA crystal lattice, as described e.g., in WO200205782A2 or WO2006128600A1).

The synthesis of both acetylsalicylic acid lysinate (LASA) and LASAG was the subject of several optimization attempts. Unfortunately, the different synthesis methods have some minor or major drawbacks. The commonly used synthesis method today involves an excess of lysine (compared to the mol of o-acetylsalicylic acid used) and the use of acetylsalicylic acid lysinate seed crystals; as described e.g., in WO200205782A2 or WO2006128600A1. One drawback of the use seed crystals is a greater risk for contamination of the final product.

Some other methods for the synthesis of acetylsalicylic acid lysinate (LASA), such as WO2011039432A1, appear to not require seed crystals, but suffer from a low yield compared to methods utilizing seed crystals (e.g. only about 70% yield compared to 90 to 95% yield in WO200205782A2).

WO2018115434A1 describes a preparation method for acetylsalicylic acid lysinate (LASA), and in particular LASA with added glycine (LASAG), which does not require the addition of seed crystals, yet still offers high yields of up to about 90 to 95%, along with an improved LASA-stability (compared to LASA without the glycine addition), and a reduced product formation time. Beneficially, the particle size of the LASAG thus obtained is smaller than with prior art products such as described in WO200205782A2 (median particle size of <100 μm compared to mean particle size of >160 μm, respectively). By controlling the particle size of LASAG, it is possible to control important parameters, such as dissolution speed (also referred to as dissolution rate), and consequently resorption speed in the body and thus onset of the pharmacological effects. A minor drawback of the method described in WO2018115434A1 is that—in order to ensure the glycine's stabilizing effect on the LASA and reduce the risk of powder segregation in the LASA+glycine solid mixture—the glycine particles have to be provided in particle sizes that approximately match those of the LASA (e.g., to prevent particle segregation during manufacturing and storage). For this purpose, WO2018115434A1 proposed to recrystallize the glycine in an acetone/water mixture prior to mixing it with the LASA.

With respect to particle size, it should also be understood that while—in theory—smaller particles sizes of ASA-particles or particles of its amino acid salts as described above could be obtained by simple grinding this approach is not at all advisable for heat-sensitive drugs such as ASA. The heat generated upon grinding would negatively impact its stability and lead to a shortened shelf-life. Therefore, processes for drug synthesis which inherently result in drug particles of the desired small particle size, such as the process described in WO2018115434A1, are advantageous over processes which would require further manipulations of the synthesized drug particles obtained.

Another disadvantage of the prior art processes described in the documents above is that they require many of their reaction steps, and in particular the crystallisation/incubation steps, to take place at temperatures below room temperature, more specifically, at cold temperatures around 5° C. or below, or 2° C. or below (e.g., 0-2° C., 0° C., or −15-0° C.). This is obviously disadvantageous in terms of time- and energy consumption.

Furthermore, LASAG particles obtained from prior art processes, such as the one described in WO2006128600A1, seem to exhibit bimodal particle size distributions (PSD), i.e., two peaks or maxima visible in PSD-graphs. This can lead to particle segregation and/or poor flow-properties of the powder bed during manufacturing steps of drug products in pharmaceutical production machines that involve powder flow of the LASAG particles. In consequence, particle segregation and poor powder flow behaviour can negatively affect dosing accuracy.

In addition, the inventors also noticed that LASAG particles of prior art processes such as in WO2006128600A1 tend to stick to glass surfaces, e.g., to the inner walls of glass vials in which ASA or it salts are commonly packaged, shipped and stored, especially when formulated as a dry powder for reconstitution. This distinct glass sticking tendency can limit visibility into the vial and thus impact handling of the vials. For instance, users run a risk of not seeing potential foreign objects and/or discolorations inside the vial that would necessitate disposal thereof; or inexperienced users may unnoticeably not dissolve and retrieve the complete intended dose from the vial upon adding water via an injection needle when drug material is sticking to the upper end of the vial, near the stopper, where the water may not reach without actively tilting or shaking the vial.

Therefore, there is still a need for a new and improved synthesis method, which i) allows for the incorporation of glycine in a manner that reduces the risk of powder segregation, or demixing, which ii) does not require seed crystals, and which iii) retains a high yield of acetylsalicylic acid lysinate (LASA), preferably ≥90%. Furthermore, there is a need for a LASAG synthesis, which allows for the easy preparation of LASAG particles with improved stability as well as defined and small particle sizes (preferably, with a median particle size below 50 μm), thus ensuring fast dissolution of the LASAG powders, e.g., upon reconstitution with water into injection solutions and/or inhalation solutions. Yet further, there is a need for a LASAG synthesis which overcomes at least some of the prior art issues mentioned above (e.g., unnecessarily time- and energy consuming processes such as processes requiring cooling for at least some of their processing steps, and/or drug material with bimodal particle size distribution (PSD) and/or distinct glass adhesion).

It is thus an object of the present invention to provide said synthesis methods, as well as stable LASAG powders with fast dissolution properties, as well as reduced glass adhesion tendencies and, preferably a unimodal particle size distribution (PSD) to facilitate safe, easy and accurate handling.

Further objects of the invention will be clear on the basis of the following description of the invention, examples and claims.

### SUMMARY OF THE INVENTION

In a first aspect, the invention relates to a method for the preparation of lysine acetylsalicylate⋅glycine (LASAG) comprising the steps of:
(a) providing a solution of acetylsalicylic acid (ASA) in ethanol;
(b) adding glycine to the solution of step a to form a suspension;
(c) providing an aqueous solution of lysine;
(d) combining the solution of step c and the suspension of step b;
(e) optionally stirring the suspension of step d;
(f) adding acetone to the suspension of steps d or e;
(g) incubating the suspension, optionally under stirring, to allow the formation of the lysine acetylsalicylate⋅glycine (LASAG) product;
(h) isolating the lysine acetylsalicylate⋅glycine (LASAG) product of step g.

In a second aspect, the invention provides a lysine acetylsalicylate⋅glycine (LASAG) obtainable by, or obtained by, the method according to the first aspect of the invention.

In a third aspect, the invention provides a lysine acetylsalicylate⋅glycine (LASAG) according to the second aspect of the invention for use as a medicine.

In a fourth aspect, the invention provides a lysine acetylsalicylate⋅glycine (LASAG) according to the second aspect of the invention for use in:
the treatment and/or prevention of viral infections in humans or animals;
the treatment and/or prevention of acute coronary syndromes (including instable angina and myocardial infarction);
the treatment of fever;
the treatment of acute moderate to strong pains (including migraine headaches).

### BRIEF DESCRIPTION OF THE DRAWINGS

FIG. 1A depicts the particle size distribution (PSD) of prior art LASAG particles as present in Bayer's commercial product Aspirin® i.v. (presumably prepared by prior art processes as described in WO2006128600A1).

FIG. 1B depicts the particle size distribution (PSD) of LASAG particles as prepared according to the present invention (‘LASAG 2’).

FIG. 2 depicts the stabilities of LASAG according to the invention and prior art LASAG as present in Aspirin® i.v. in aqueous solution over time for solutions kept either at room temperature (i.e., as used herein, a temperature in the range of 20±5° C., or 15-25° C., as defined e.g., by the European Pharmacopoeia or by the 2003 WHO guidance ‘Guidelines for the Storage of Essential Medicines and Other Health Commodities’), or in cold storage (i.e., as used herein, a temperature in the range of 5±3° C. or 2-8° C.); expressed as the increase of the salicylic acid (SA) content in weight-percent in the stored LASAG-solution, as determined by HPLC.

### DETAILED DESCRIPTION OF THE INVENTION

The inventors found an improved method for the production of lysine acetylsalicylate⋅glycine (LASAG) which provides very high yields (≥90%) without the need for seed crystal addition and at reduced product formation times and reduce energy consumption. The method further allows for the production of LASAG particles with defined and small particle sizes, a unimodal particle size distribution, as well as with a lower tendency for adhesion to glass surfaces. All of these benefits can be achieved without compromises to the stability of the LASAG particles.

In a first aspect, the invention relates to a method for the preparation of lysine acetylsalicylate⋅glycine (LASAG) comprising the steps of:
(a) providing a solution of acetylsalicylic acid (ASA) in ethanol, said solution optionally being filtered in a step a₁;
(b) adding glycine to the solution of step a to form a suspension;
(c) providing an aqueous solution of lysine;
(d) combining the solution of step c and the suspension of step b;
(e) optionally stirring the suspension of step d;
(f) adding acetone to the suspension of steps d or e;
(g) incubating the suspension, optionally under stirring, to allow the formation of the lysine acetylsalicylate⋅glycine (LASAG) product;
(h) isolating the lysine acetylsalicylate⋅glycine (LASAG) product of step g.

Acetylsalicylic acid, within the context of the present invention, preferably refers to o-acetylsalicylic acid; i.e., unless where specified other ‘acetylsalicylic acid’ or ‘ASA’ refer to the ortho-form thereof. Same applies to the respective lysine acetylsalicylate⋅glycine (LASAG) obtained from said acetylsalicylic acid.

The solutions of steps (a) and (c) should comprise sufficiently pure compounds and be based on pharmaceutical grade solvents. Same applies to the acetone added in step (f). The compounds acetylsalicylic acid, and lysine, as well as the glycine added in step (b) are preferably at least substantially pure, more preferably at least of pharmaceutical grade purity, most preferably essentially free of impurities.

In one embodiment of the method, the lysine acetylsalicylate⋅glycine (LASAG) is obtained in the form of a co-crystal. As used herein, this means that the glycine is embedded, or incorporated, within the crystal lattice of the lysine acetylsalicylate (LASA), or that the two crystals are otherwise conjoined or intergrown with one another (e.g., glycine attached via hydrogen bonds to the LASA, or LASA precipitated around the glycine) to form a LASAG compound, in which the glycine becomes inseparable from the LASA. For reasons of brevity, this may also be referred to herein as ‘inner-crystalline’ or ‘internal’ glycine (as opposed to an ‘external’ glycine as it would be present when glycine is added after the synthesis, or crystallisation, of the LASA particles, e.g., in solid mixtures of glycine and LASA, as described e.g., in WO2018115434A1).

It is important for the method that the molar amount of acetylsalicylic acid exceeds the molar amount of lysine in the final mixture; thus in one embodiment of the method, the acetylsalicylic acid is used in excess compared to lysine. For instance, in a specific embodiment, acetylsalicylic acid is used in at least 1.02-fold molar excess compared to lysine, preferably at least 1.04-fold, and more preferably, at least 1.05-fold. In a further specific embodiment, the acetylsalicylic acid (ASA) and lysine are used at a molar ratio in the range of 1:0.99-0.70, or 1:0.98-0.80, or 1:0.97-0.90, or 1:0.96-0.92; e.g., at a molar ratio of 1:0.9, or at a molar ratio of 1:0.95. In one exemplary embodiment, about 1.5 kg ASA (about 0.833 mol ASA) was mixed with about 1.3 kg D,L-lysine monohydrate (about 0.791 mol lysine). In this regard, the preparation method according to the invention differs from prior art processes such as described in WO200205782A2 or WO2006128600A1 which use an excess of lysine compared to the ASA (e.g., an ASA-lysine molar ratio from 1:1.05 to 1:1.5).

In one embodiment of the method, no seed crystals are added during the preparation. In particular, no seed crystals comprising, or consisting of, acetylsalicylic acid (ASA), lysine acetylsalicylate (LASA), or lysine acetylsalicylate⋅glycine (LASAG) are added during the preparation. This differentiates the preparation method according to the invention from prior art processes such as described in WO200205782A2 or WO2006128600A1 which require the use of seed crystals to obtain higher yields.

In a specific embodiment of the method, the acetylsalicylic acid is used in excess compared to lysine, and no seed crystals are added during the preparation. In a more specific embodiment of the method, the acetylsalicylic acid is used in excess compared to lysine, and no seed crystals comprising, or consisting of, acetylsalicylic acid (ASA), lysine acetylsalicylate (LASA), or lysine acetylsalicylate⋅glycine (LASAG) are added during the preparation.

The solutions of steps a and c might be pretreated before use. Pretreatment comprises any treatment before the use of the solutions in the method and may involve, for instance, that a solution is heated or cooled, filtered and/or irradiated prior to use in the method; e.g., the ASA-solution of step a being pretreated prior to the glycine addition in subsequent step b, or the lysine-solution of step c being retreated prior to combining it in subsequent step d with the ASA-glycine suspension of step b. In one embodiment, at least one of the solutions of steps a and c has been heated or cooled, filtered and/or irradiated prior to use in the method. The same applies mutatis mutandis to the suspension of step b, with the exception of pretreatment by filtration, since filtration steps would remove the added glycine powder from the suspension.

In one embodiment of the method, the ethanolic ASA-solution of step a is prepared freshly prior to the subsequent steps. When referring herein to ethanol as a solvent, a dehydrated ethanol, denatured with 2% cyclohexane, is typically employed; yet, absolute ethanol (100% V/V), or dehydrated ethanol denatured with substances other than cyclohexane are considered equally suited. Ethanol 96% V/V can also be used; in this case, it is recommended, though, to counterbalance its higher inherent water-content by using the respective lower amounts of water in other steps of the method according to the first aspect of the invention. In a further embodiment of the method, the ethanolic ASA-solution of step a is prepared at elevated product temperatures, such as about 30±5° C. (i.e., using mild warmth to aid the ASA-dissolution), optionally in a reaction vessel equipped with a temperature-control jacket and at a jacket-temperature of about 30° C. In a specific embodiment of the method, the ethanolic ASA-solution of step a is prepared freshly and at elevated product temperatures, such as about 30±5° C., prior to the subsequent steps. In a more specific embodiment of the method, and especially where elevated product temperatures such as about 30±5° C. were employed to aid ASA-dissolution, the ethanolic ASA-solution of steps a or a₁ is cooled down to room temperature (i.e., as used herein, a temperature in the range of 20±5° C.) prior to step b, optionally under stirring to aid cool-down.

In one embodiment of the method, the ethanolic ASA-solution of step a comprises, or consists of, at least about 8 wt.-% acetylsalicylic acid, and ethanol. In a specific embodiment of the method, the ethanolic ASA-solution of step a comprises, or consists of, about 8 to 20 wt.-%, or about 12 to 19 wt.-%, or about 14 to 18 wt.-% acetylsalicylic acid, and ethanol, based on the final weight of the ASA-solution, e.g., about 16.7 wt.-%. For instance, in one exemplary embodiment, 1.5 kg acetylsalicylic acid may be dissolved in 9.5 L ethanol (≥96% V/V) under stirring; optionally using elevated product temperatures such as about 30±5° C. to aid ASA-dissolution. This differentiates the preparation method according to the invention from prior art processes such as described in WO200205782A2 or WO2006128600A1 which suggest lower ASA-concentrations in the range of 1 to 10% wt.-%, preferably 6 to 8 wt.-%.

As mentioned above, the solutions of steps a and c might be pretreated before their use. In an optional embodiment of the method, the ethanolic ASA-solution of step a is filtered in a step a₁, optionally sterile-filtered and/or filtered for pyrogen removal, prior to any of the subsequent steps, in particular, before the addition of the glycine, since the latter would not be soluble in the ethanolic ASA-solution and thus hinder the filtration step.

In one embodiment of the method, the glycine is added to the ethanolic ASA-solution of steps a or a₁ as a dry powder. In that regard, the preparation method according to the invention differs from prior art processes such as described in WO200205782A2 or WO2006128600A1 which teach the use of an aqueous glycine solution or suspension. Optionally, the glycine powder added is a recrystallized glycine obtained by dissolving glycine in water, adding acetone to the glycine solution, and stirring the mixture until a glycine precipitate is obtained, such as described e.g., in WO2018115434A1.

In a further embodiment of the method, the glycine in step b is added to the ethanolic ASA-solution of steps a or a₁ at a weight ratio of acetylsalicylic acid to glycine in the range of 1:0.28-0.09, or 1:0.24-0.14; e.g., at a weight ratio of 1:0.21, or 1:0.19, or 1:0.17. For instance, in one exemplary embodiment, 279 g glycine powder is added under stirring to a solution of 1.5 kg ASA dissolved in 9.5 L ethanol (≥96% V/V), with the stirring speed chosen in such a way that a homogenous, agglomerate-free dispersion of the glycine powder is possible, while at the same time avoiding unnecessarily fast stirring speeds that could result in excessive ethanol evaporation.

In a yet further embodiment of the method, the glycine in step b is added to the ethanolic ASA-solution of steps a or a₁ in an amount so as to yield a glycine content of about 8 to 12 wt.-%, or 9 to 11 wt.-%, or about 10 wt.-% based on the final isolated LASAG product of step h.

In step c of the method an aqueous lysine solution is provided. Preferably, lysine is used in the form of the free base, including e.g., stereo-isomers thereof (L- or D-lysine), its racemic form (D,L-lysine), or its solvates, such as hydrates like lysine monohydrate. While it is possible to use lysine in salt form (e.g., a lysine hydrochloride), it is preferred to use lysine in its free base form, e.g., as lysine monohydrate. When referring to any ratios or concentrations of lysine herein, though, these are based on the lysine only, irrespective of whether or not, for instance, a monohydrate has been used.

In one embodiment of the method, the aqueous solution of lysine provided in step c is prepared from lysine monohydrate, optionally L- or D,L-lysine monohydrate. In a specific embodiment, the aqueous solution of lysine provided in step c consists of lysine monohydrate, optionally L- or D,L-lysine monohydrate, and water.

The solution comprising lysine is preferably of higher concentration than the solution comprising acetylsalicylic acid. In one embodiment, the aqueous solution of lysine provided in step c comprises, or consists of, at least about 41 wt.-% lysine, and water, with the lysine optionally being provided in the form of its L- or D,L-lysine monohydrate. In a specific embodiment, the aqueous solution of lysine provided in step c comprises, or consists of, about 41 to 55 wt.-% lysine, or about 40 to 50 wt.-% lysine, or about 44 to 48 wt.-% lysine, and water, based on the final weight of the lysine-solution; e.g., 45.7 wt.-% lysine. For instance, in one exemplary embodiment, an aqueous lysine solution may be prepared from about 1.3 kg D,L-lysine monohydrate and 1.25 L water. This differentiates the preparation method according to the invention from prior art processes such as described in WO200205782A2 or WO2006128600A1 which suggest the use of aqueous lysine solutions containing only 10-40 wt.-% lysine, preferably only 20-30 wt.-%. Due to the higher lysine concentrations used in the method according to the invention, pure water (i.e., without added further solvents such as ethanol or acetone) is the preferred dissolution medium for the preparation of the lysine solution. Furthermore, the use of higher lysine concentrations advantageously allows to significantly reduce the amounts of ethanol and/or acetone used in the preparation method.

As mentioned above, the solutions of steps a and c might be pretreated before their use. In an optional embodiment, the aqueous lysine-solution of step c is filtered in a step c₁, optionally sterile-filtered and/or filtered for pyrogen removal, prior to the subsequent steps.

The combination step d can be performed in any suitable way. In one of the preferred embodiments, the ASA-glycine-suspension of step b and the aqueous lysine solution of step c are provided at room temperature (20±5° C.) for step d, or, where applicable, are allowed to cool down to room temperature (optionally under stirring), prior to step d. In a further preferred embodiment, the ASA glycine-suspension of step b and the aqueous lysine solution of step c are combined slowly and still at room temperature in step d, while optionally stirring the forming mixture (step e). Ideally, the mixture will start to crystallize during the mixing process, as is then indicated, for instance, by the suspension growing thicker. In other words, at least method steps a-d, or steps a-e, do not require cooling to temperatures below room temperature, or more specifically no cooling to 15° C. or below, or 10° C. or below, and in particular no cooling to temperatures near or below freezing point at 0° C., such as about-5 to 5° C. In this regard, the preparation method according to the invention differs from prior art processes such as described in WO2006128600A1 which require cooling to −5-10° C., preferably to 0-5° C. (e.g., 2° C. in Example 1), as well as extended stirring of at least 1 h, before adding the glycine to their cooled ASA and lysine solution.

Moreover, as mentioned above, the mixture being formed in steps d or e usually starts to crystallize during the mixing process already, i.e., forming at least an initial precipitate, which is noticeable by the suspension growing thicker. No seed crystals are needed or added for this purpose (in particular no seed crystals comprising, or consisting of, acetylsalicylic acid (ASA), lysine acetylsalicylate (LASA), or lysine acetylsalicylate⋅glycine (LASAG)), and usually the first precipitate forms within 10 minutes or less, e.g., within 5 minutes.

In one embodiment, the combination step d is performed at ambient pressure.

In one of the preferred embodiments of the method, the combination step d is performed by adding the aqueous lysine-solution of step c to the ASA-glycine-suspension of step b, preferably while the forming mixture is stirred. For instance, in one exemplary embodiment, an aqueous lysine solution may be prepared from about 1.3 kg D,L-lysine monohydrate and 1.25 L water, and then added to an ethanolic ASA-glycine suspension (e.g., 1.5 kg dissolved ASA and 279 g glycine powder in 10.5 L ethanol (≥96% V/V)). In this example, the lysine-solution may, for instance, be added at a speed of about 0.9 kg lysine solution per minute and kilogram ASA-glycine suspension.

It is generally preferred that the volume of the ethanolic ASA-solution of step a exceeds the volume of the aqueous lysine solution of step c. Preferably, the volume of the ethanolic ASA-solution of step a is at least about 2 times, preferably at least about 3 times, more preferably at least about 4 times as large as the volume of the aqueous lysine solution of step c. In a specific embodiment, the volume of the ASA-glycine-suspension of step b exceeds the volume of the aqueous lysine-solution of step c.

Preferably, the ASA-glycine-suspension of step b and the aqueous lysine solution of step c are combined within less than one hour, more preferably within less than 30 minutes, more preferably within less than 15 minutes, even more preferably within less than 10 minutes, most preferably within less than 5 minutes.

Typically, while combining the ASA-glycine suspension of step b, and the aqueous lysine-solution of step c in step d, the mixture of the two is stirred as indicated in step e to ensure its homogeneity prior to the addition of acetone in step f. Where available, alternative means of mixing other than stirring, such as air jets or the like, can be applied as well.

In one embodiment of the method, the suspension of steps d or e (i.e., the mixture of the ASA-glycine suspension of step b, and the aqueous lysine-solution of step c) is stored, and optionally stirred, for less than 24 hours prior to the addition of the acetone in step f, preferably less than 12 hours, or less than 6 hours, or less than 3 hours; more preferably less than 1 hour. For instance, in one exemplary embodiment, the suspension of step e is stirred at about 150 rpm for about 45 minutes before adding acetone to it.

After the combination step d, and optional stirring in step e, in step f, acetone is added to the suspension of steps d or e. In one embodiment, the amount of acetone added is lower than that of the suspension of step e. In a more specific embodiment, the amount of acetone added is also lower than that of the ethanolic ASA-solution of step a; or in other words, the volume of the ethanolic ASA-solution in step a exceeds the volume of the acetone added in step f. This differentiates the preparation method according to the invention from prior art processes such as described in WO200205782A2 or WO2018115434A1 which explicitly require an excess volume of acetone over the volume of the ASA-solution.

The addition of acetone should result in a supersaturated mixture, which leads to improved and faster crystallization with higher yields. Accordingly, the amount of acetone used should be sufficient to ensure supersaturation of the mixture. In a preferred embodiment of the invention the acetone is added after crystallization has started, i.e., after initial precipitate has formed. Usually the first precipitate should form in steps d or e, as mentioned above, without the use of seed crystals within 10 minutes or less. In several embodiments of the invention, the first precipitate forms within five minutes.

In one embodiment of the method, at least method steps b to g are performed at room temperature (20±5° C.), or below. However, working at room temperature is obviously advantageous and thus preferred herein in so far as it is both convenient and efficient since it saves the energy for cooling while still providing a high yield of 95% or higher. Thus, in one of the preferred embodiments, at least the incubation step g is performed at room temperature (20±5° C.). This differentiates the preparation method according to the invention from prior art processes such as described in WO2011039432A1 or WO2006128600A1 which require lower temperatures in the range of −15 to +15° C. or 0-5° C., respectively, for the incubation step g after the addition of the acetone. WO2006128600A1 expressly highlights the importance of performing the incubation- and crystallisation of the prior art product in a narrowly defined temperature range of only about 0-2° C., and advises that the temperature should not exceed 5° C., preferably not exceed 3° C. Therefore, the preparation method according to the invention clearly offers advantages over the prior art processes, being both easier and more energy efficient. The addition of acetone in step f is preferably performed at the same temperature as steps d and e, or at the same temperature as the incubation step g. Thus, in one of the preferred embodiments, step f is performed at room temperature. This means that in one of the preferred embodiments, all of method steps a-g are performed at room temperature, or, in other words, all of method steps a-g do not require cooling to temperatures below room temperature, or more specifically no cooling to 15° C. or below, or 10° C. or below, and in particular no cooling to temperatures near or below freezing point at 0° C., such as about −5 to 5° C.

As mentioned above, not requiring cooling below room temperature is advantageous in terms of time- and energy consumption of the synthesis process. That being said, though, the benefit of being able to perform the method according to the invention, and in particular steps a-g thereof, at room temperature should not be misunderstood to mean that it must or can only be performed at room temperature. Lower temperatures, such as the ones suggested in the prior art (e.g., about −15-5° C.) could still be employed, especially for the mixing and incubation steps d to g, without this choice negatively impacting the yield and/or the particle properties of the LASAG particles obtained, such as particle size, particle size distribution, or glass adhesion behaviour. In other words, performing the process according to the first aspect of the invention, in the order and with the components as described herein, yet choosing to work at cold temperatures such as 10° C. or below, or 5° C. or below, would not fall outside the scope of the present invention.

After the addition of acetone, the suspension of step f (i.e., the mixture of the ASA-glycine suspension of step b, the aqueous lysine-solution of step c, and the added acetone of step f) should be allowed to incubate to allow formation, or completion of formation, of the lysine acetylsalicylate⋅glycine (LASAG) co-crystals. As mentioned above, the incubation step g is preferably performed at room temperature (20±5° C.), or below, optionally under stirring. The suspension can be incubated, for as long as considered necessary to obtain a high yield, typically for at least about 30 minutes. However, one of the advantages of the method according to the invention is the short incubation time in step g. High yields of product are achieved in three hours of incubation time or less. Thus, in one embodiment of the invention, the suspension is incubated, optionally under stirring, for about three hours or less, preferably for about two hours or less, more preferably for about one hour or less. In one of the preferred embodiments, the suspension is incubated for about 30-60 minutes.

Longer stirring times such as stirring overnight can, of course, be used as well if considered more suitable, either in terms of product stability, and/or for the general timing of the individual process steps. For instance, in one exemplary embodiment, the suspension is stirred for 17±1 hours at about 20° C. to allow for the formation and precipitation of the lysine acetylsalicylate⋅glycine (LASAG) particles. These particles were found to be particularly stable. In a further embodiment of the method, the suspension of step g is stored, and optionally stirred, for 24 hours or less, preferably for 20 hours or less, and more preferably for 18 hours or less before retrieving; or isolating, the LASAG particles in step h.

After incubation in step g, the precipitated product is isolated in step h. Any method which allows separation of the precipitated product from the liquids is suitable. In one embodiment of the method, the product isolation step h is performed by filtration. In an alternative embodiment of the method, the product isolation step h is performed by centrifugation. Optionally, the product isolation step h is performed by filtration and centrifugation, i.e., both techniques are employed for isolation.

In one embodiment, the method additionally comprises a step i of washing the isolated product, e.g., to remove impurities. In a specific embodiment, the washing step i involves washing the isolated product with ethanol and/or acetone. In a further specific embodiment, the washing step i involves washing the isolated product with ethanol, preferably washing it with ethanol twice, optionally three times. In a further specific embodiment, the washing step i involves washing the isolated product with ethanol, followed by washing the isolated product with acetone; optionally repeating these washing steps.

The isolation step h and the optional washing step i are preferably performed at the same temperature, or a similar temperature, as the incubation step g; i.e., typically, at room temperature (20±5° C.), or below. In the less common case, where a different temperature is used, the mixture containing the to-be-isolated, optionally to-be-washed, LASAG product, and/or said product as such, may or may not adapt to the temperature used after incubation; for instance, if the incubation step g was performed at 5° C. (even though not required), and the isolation at 20° C., the mixture and/or product may or may not warm up to 20° C. before the isolation step h is finished.

After isolating and washing the product, the product may optionally be dried. Within the context of the invention the term drying refers to the removal of solvent residues, preferably the removal of excess water, ethanol and acetone. The solvents may be removed by any suitable method, with drying under reduced pressure until reaching ≤15 mbar, optionally at a temperature of about 30° C., being one of the preferred means of drying.

In one embodiment, the method is performed under sterile conditions. For instance, as mentioned above, the ethanolic acetylsalicylic acid solution of step (a) and/or the aqueous solution of lysine of step (c) may be pretreated in order to sterilize the solution(s) before use, optionally by sterile and/or pyrogen removal filtration.

Alternatively, the method is performed under non-sterile conditions and comprises an additional step of sterilizing the product obtained in steps h or i, typically by irradiation, optionally gamma-irradiation. Radiation is a preferred means of sterilization for the obtained LASAG particles, since the raw material is sensitive to heat and thus not suited for heat sterilization methods.

The method is particularly suitable for producing LASAG particles with a defined particle size. In one embodiment of the method, for instance, the particles of the product obtained in steps h or i have a median particle size (D50) of less than 40 μm, or less than 30 μm, or less than 20 μm. This particle size results inherently from following the method as described herein, and does not require further manipulations of the synthesized drug particles obtained, such as grinding to this small particle size, which could negatively impact the heat-sensitive drug.

Unless mentioned otherwise, the particle size values provided herein (either measured or calculated/derived from measured values) refer to particle diameters and were determined using a laser diffraction device, and its related evaluation software (here, for instance, a Mastersizer® device from Malvern Instruments Ltd.); all laser diffraction measurements complied with ISO-13320 standards. Where referring to the particle size measurements, all percentages provided herein (such as ‘at least 90% of the particles have a particle size of . . . ’) are to be understood as volume-percentages.

In a further embodiment of the method, at least 90% of the particles obtained in steps h or i have a particle size (D90) of less than 65 μm, or less than 55 μm, or less than 45 μm.

In a specific embodiment, the particles of the product obtained in steps h or i have a median particle size (D50) of less than 40 μm, and at least 90% of the particles have a particle size (D90) of less than 65 μm; or a D50-value of less than 30 μm, and a D90-value of less than 55 μm; or a D50-value of less than 20 μm, and a D90-value of less than 45 μm.

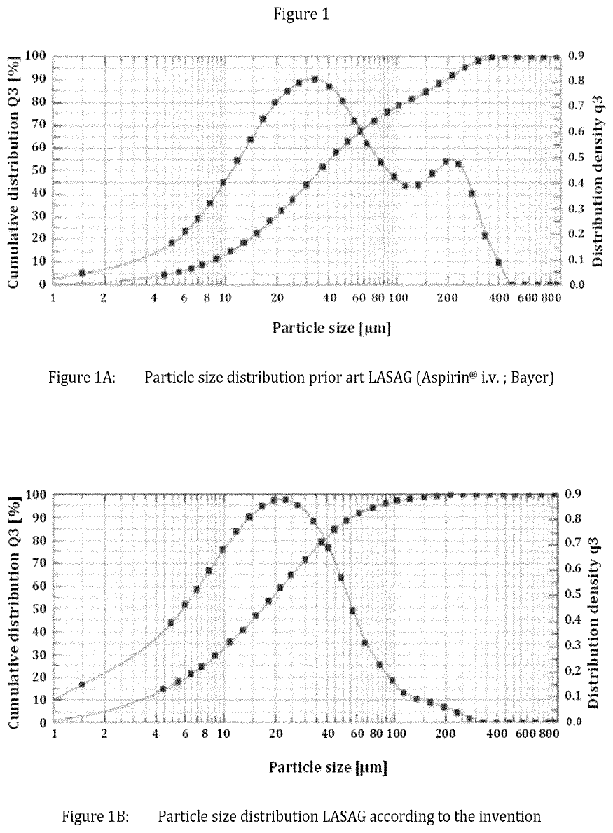

In one embodiment, the particles of the product obtained in steps h or i exhibit a D90/D50 ratio of 3.7 or less, or 3.4 or less, or 3.1 or less (or, in other words, a narrow particle size distribution). In a further embodiment of the method, the particles of the product obtained in steps h or i exhibit a unimodal particle size distribution (PSD), ie., only one peak or maximum visible in PSD-graphs such as depicted in FIG. 1A or 1B.

A unimodal particle size distribution is beneficial insofar as it helps reduce particle segregation and/or poor flow-properties of the powder bed during manufacturing steps of drug products in pharmaceutical production machines that involve powder flow of the LASAG particles. Reduced particle segregation and improved powder flow behaviour consequently also helps to improve dosing accuracy. This advantageous, homogenous powder flow behaviour applies even more so to particles, such as the LASAG described herein, which exhibit not only a unimodal particle size distribution but one that furthermore comes with a D90/D50 ratio of 3.7 or less, or 3.4 or less, or 3.1 or less.

The particle size distribution (PSD) data of various LASAG particles are also shown in Table 1 and FIGS. 1A and 1B. Where available, three or more batches of the substances were tested, and mean values of their PSD-data obtained.

In one embodiment, the method exhibits a yield of at least 90%, or at least 92%, or at least 94%, or at least 96%, such as 97%. Said yield is based on the amounts of lysine and glycine used in the method according to the first aspect of the invention. In a specific embodiment, the method exhibits a yield in the range of 90% to 100%, or 94% to 99%, or 96% to 98%.

In an optional embodiment of the method, the aqueous solution of lysine provided in step c further comprises dissolved glycine.

One of the main advantages of the method according to the first aspect of the invention is that i) it allows for the incorporation of glycine in a manner that reduces the risk of powder segregation, or demixing, of LASA and glycine; that ii) no seed crystals are necessary to achieve high purity of the product after a short product formation time, even in industrial scale; and that iii) it provides a high yield of lysine acetylsalicylate⋅glycine (LASAG), preferably ≥90%. Moreover, as mentioned above, the method according to the first aspect of the invention also does not require cooling below room temperature to achieve these yields, and results in LASAG particles offering a number of beneficial properties (such as inherently small median particle size below 40 μm with higher dissolution speeds, narrow, unimodal particle size distribution, reduced glass adhesion tendencies, and good stability performance). Other methods used in industrial scale either require the use of seed crystals and/or cooling well below room temperature; and/or they result in significantly lower yields than the method of the invention.

In a second aspect, the invention provides a lysine acetylsalicylate⋅glycine (LASAG) obtainable by, or obtained by, the method according to the first aspect of the invention. For brevity, these LASAG particles may be referred to herein as ‘LASAG 2’.

All embodiments, including all specific or preferred embodiments, as described above in connection with the method of the first aspect of the invention also apply to the LASAG according to this second aspect of the invention, and its particles. For instance, in one embodiment, the LASAG according to the second aspect of the invention is in the form of a co-crystal, i.e., the glycine is embedded in, or incorporated within, the crystal lattice of the lysine acetylsalicylate (LASA), or otherwise conjoined or intergrown with the LASA; herein called inner-crystalline glycine.

As mentioned above, the LASAG particles obtainable by, or obtained by, the method according to the first aspect of the invention have a median particle size (D50) of less than 40 μm, or less than 30 μm, or less than 20 μm (see e.g., Table 1). In other words, these LASAG particles are smaller than the prior art LASAG particles with inner-crystalline glycine described in WO200205782A2, for which a mean particle size of >160 μm, preferably >170 μm, was considered beneficial, and 60%, preferably 70%, of the particles were in the 100 to 200 μm size range). They are also smaller than the prior art LASAG particles with inner-crystalline glycine described in WO2006128600A1, for which a mean particle size of <100 μm, preferably <70 μm is disclosed and which are presumably employed in the currently available commercial Aspirin® i.v. product, (see e.g., Table 1).

While the particle size differences portrayed in Table 1 (e.g., LASAG 2 compared to a LASAG as present in Aspirin® i.v. and presumably prepared by the process described in WO2006128600A1), the inventors found a clinically relevant difference in dissolution speed, as illustrated in Example 2 below.

In a further embodiment, at least 90% of the LASAG particles according to the second aspect of the invention have a particle size (D90) of less than 65 μm, or less than 55 μm, or less than 45 μm (see e.g., Table 1). This means that these LASAG particles means are smaller than those described in e.g., WO2006128600A1, and also smaller than the applicant's prior art LASAG particles such as described in WO2018115434A1, with D90-values of about 200 μm and about 100 μm, respectively, compared to only about 50 μm for the LASAG of the present invention; see e.g., Table 1.

In a specific embodiment, the LASAG particles have a median particle size (D50) of less than 40 μm, and at least 90% of the particles have a particle size (D90) of less than 65 μm; or a D50-value of less than 30 μm, and a D90-value of less than 55 μm; or a D50-value of less than 20 μm, and a D90-value of less than 45 μm.

As mentioned above, by controlling the particle size of the LASAG, it is possible to control parameters such as the dissolution speed (also referred to as dissolution rate). Due to the larger surface-to-volume ratio of smaller particles, the LASAG according to the second aspect of the invention dissolves noticeably quicker than, for instance, larger sized prior art LASAG particles, such as those described in WO2006128600A1 (and as presumably used in the commercially available Aspirin® i.v.). For instance, using the same technique of injecting 5 mL of water for injection purposes to 8 mL glass vials through the top-seal, each vial filled with 1000 mg LASAG, and then shaking or swirling the contents manually until complete dissolution (no residual solids visible to the human eye), it was noticed that the LASAG particles according to the second aspect of the invention (‘LASAG 2’) required less time and/or less manual shaking/swirling movements until complete dissolution than, for instance, the prior art LASAG particles as present in Aspirin® i.v.; namely, about 15-30 seconds with only 3-5 shaking/swirling motions for the ‘LASAG 2’ versus about 40-60 seconds and using 8-10 shaking/swirling motions for Aspirin® i.v. (see e.g., Example 2). The product insert of Aspirin® i.v. (status July 2019) even suggests under item “6.6. Special precautionary measures” that the reconstituted solution should preferably be filtered with a 5 μm filter before its application, as such indicating potential issues with (quick) dissolution. Especially, when considering that LASAG is inter alia employed in the treatment of myocardial infarction, i.e., an acute health crisis where time is of critical importance, a faster, more readily occurring dissolution of the LASAG used, can represent a clinically relevant advantage.

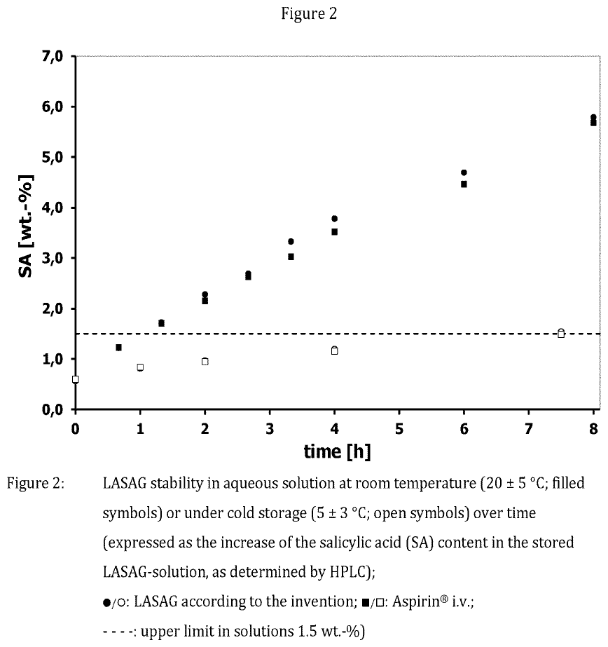

Advantageously, the faster dissolution properties of the LASAG particles according to the second aspect of the invention (‘LASAG 2’) do not influence the compound's stability in water: the ‘LASAG 2’ particles exhibit equal stability in aqueous solution as the prior art LASAG particles present in the Aspirin® i.v. product, as is depicted in terms of the LASAG-degradation related formation of salicylic acid in FIG. 2. The respective method to determine the stability in aqueous solution is described in Example 5 below.

In one embodiment, the LASAG particles exhibit a D90/D50 ratio of 3.7 or less, or 3.4 or less, or 3.1 or less. In other words, the LASAG particles according to the second aspect of the invention exhibit a narrower particle size distribution (PSD) than prior art LASAG particles; for instance, the particles in the tested Aspirin® i.v. product (presumably prepared according to WO2006128600A1) exhibits a D90/D50 ratio of ≥5.5, and the applicant's prior art LASAG particles (prepared according to WO2018115434A1) exhibit a D90/D50 ratio of ≥3.8; see e.g., Table 1.

In a further embodiment, the LASAG particles exhibit a unimodal particle size distribution. This distincts them from the prior art LASAG particles as found, for instance, those present in the commercially available Aspirin® i.v. which exhibits a double peak, or in other words a bimodal particle size distribution (see e.g., FIGS. 1A and 1B).

Both, lower D90/D50 ratios and/or unimodal particles size distributions, indicate more homogenously sized LASAG particles, which is beneficial in so far as it avoids, or at least reduces, the risk for particle segregation during e.g., filling- and dosing steps (e.g., in feeding hoppers), shipping and storage. Without wishing to be bound by theory, the inventors believe that this more homogenous particle size distribution of the LASAG according to the second aspect of the invention (‘LASAG 2’) is related the formation of overall finer crystals compared to the coarser prior art LASAG crystals found in the commercial Aspirin® i.v. product. This difference was also found, for instance, in Xray powder diffraction (XRPD) data where the Aspirin® i.v. particles showed sharper glycine-related reflexes than the LASAG 2. No amorphous fractions were identified in the XRPD-data obtained for the LASAG 2 crystals, indicating a very well crystallized product.

In one embodiment, the LASAG particles exhibit low adhesion to glass surfaces, for instance, a glass adhesion of 0.250 mg/cm² or less, preferably 0.200 mg/cm² or less. This can be tested, for instance, by filling a tared 8 mL glass vial, such as those commonly used as containers for powders for reconstitution, with 1000 mg of the LASAG particles to be tested, closing the vial, and manually rotating it around all its axes to properly disperse the LASAG particles and allow them to coat the inner glass surface; this can be repeated, e.g., over the course of five days. After opening the vial and emptying out any non-adhered powder by simple gravitational spill, the vial with the adhered particles inside is weighed again, to thereby gravimetrically determine the weight of the adhered particles. Low glass adhesion facilitates handling of the vials (e.g., better visibility into the vial), and ensures that the complete intended dose is dissolved upon adding water to the vial (rather than, for instance, some material sticking to the upper end of the vial, near the stopper, where the water, that is added into the vial via injection through a needle, may not reach).

In one embodiment, the LASAG particles exhibit a specific surface area, as measured via BET gas adsorption technique, which is 1.50 m²/g or higher, or 1.75 m²/g or higher, or 2.00 m²/g or higher, or 2.25 m²/g or higher. For instance, in one exemplary embodiment, the LASAG particles exhibit a specific surface area of 2.4±0.1 m²/g. In contrast, the prior art LASAG present in Aspirin® i.v. exhibited a specific surface area of only 1.1±0.1 m²/g in the same BET-measurement. Without wishing to be bound by theory, it seems possible that not only the smaller median particle size of the LASAG according to the present invention is responsible for its faster dissolution but also its higher specific surface area.

In one embodiment, the LASAG particles exhibit a density of 1.450 g/cm³ or higher, or 1.460 g/cm³ or higher, or 1.470 g/cm³ or higher.

In one embodiment, the LASAG particles exhibit a residual moisture of 0.15 wt.-% or lower; for instance, a residual moisture of 0.10 wt.-%. This is well within the respective specifications of similar prior art LASAG products such as Aspirin® i.v. (requiring a residual moisture content of ≤0.3 wt.-%), and advantageously improves the storage stability of the ‘LASAG 2’ particles in dry state, as shown in e.g., Table 2.

At 5±3° C. (cold storage), the samples do not exceed the upper degradation limit of ≤1.5 wt.-% salicylic acid for more than 60 months, i.e., 5 years (see e.g., the extrapolated value of 70.9±3.5 months in Table 2). After continuation of the cold storage stability study for a further 12 months, this stability finding is reconfirmed, with the extrapolated value being increased to from 70.9±3.5 months to 200±5 months.

In a third aspect, the invention provides a lysine acetylsalicylate⋅glycine (LASAG) according to the second aspect of the invention for use as a medicine. For this purpose, the lysine acetylsalicylate⋅glycine (LASAG) according to the second aspect of the invention (or in other words, the LASAG particles obtainable by, or obtained by, the method according to the first aspect of the invention) may be prepared in any way needed to access the desired route of administration. For instance, in some embodiments, the LASAG may be compressed into tablets or filled into capsules, typically together with known pharmaceutically acceptable excipients. In alternative embodiments, the LASAG particles may be used in powdered form, for instance, in vials containing the dry LASAG-particles for reconstitution with added water for injection purposes.

In a fourth aspect, the invention provides a lysine acetylsalicylate⋅glycine (LASAG) according to the second aspect of the invention for use in:
the treatment and/or prevention of viral infections in humans or animals;
the treatment and/or prevention of acute coronary syndromes (including instable angina and myocardial infarction);
the treatment of fever;
the treatment of acute moderate to strong pains (including migraine headaches).

The following examples serve to illustrate the invention, however should not to be understood as restricting the scope of the invention.

### EXAMPLES

### Example 1—Preparation of LASAG According to the Invention (‘LASAG 2’; Lab Scale)

In a reaction vessel equipped with a temperature controlled jacket, an amount of 1.5 kg acetylsalicylic acid (ASA) is dissolved in 9.5 L ethanol (here, dehydrated ethanol, denatured with 2% cyclohexane) at elevated product temperatures, i.e., employing mild warmth (here, a jacket-temperature of about 30° C.).

The freshly prepared, filtered-optionally sterile-filtered-ASA-solution is then cooled down to a product temperature of 20±5° C. while stirring, prior to adding and homogeneously suspending 279 g of powdered glycine in the ethanolic ASA-solution under stirring (here, e.g., 100 rpm), thereby forming a homogenous ASA-glycine suspension. The transfer container is rinsed with 1.0 L ethanol to ensure complete transfer of the intended amount of powdered glycine into the ASA-solution.

Prior to adding lysine to this suspension, the stirring speed is increased slightly (here, e.g., from 100 rpm to 150 rpm), and the product temperature reduced by approx. 5° C. to approx. 15±5° C. Then, a freshly prepared, filtered-optionally sterile-filtered-solution of 0.95 mol equivalents lysine (based on the acetylsalicylic acid) in 1.25 L demineralized water is added to the suspension in the reaction vessel over the course of a few minutes (approx. 2-3 min.), and the transfer container rinsed with another 0.25 L water to ensure complete transfer of the intended amount of lysine into the ASA-glycine suspension. In this experiment, the lysine equivalents were provided in the form of 1282 g D,L-lysine monohydrate (about 1.3 kg) with a hydrate water content of about 9.8 wt.-%; i.e., about 1156 g lysine together with about 126 g crystal water, dissolved in 1.25 L water.

After the addition of D,L-lysine monohydrate is complete, the suspension is stirred for approx. 45 minutes at 150 rpm before adding 7.5 L acetone to it. The suspension is stirred for 17±1 hours at a jacket-temperature of 20° C. to allow for the formation and precipitation of the lysine acetylsalicylate⋅glycine (LASAG) particles.

The resulting suspension is filtered off, the filter cake is washed twice with ethanol and twice with acetone. The solid is dried under reduced pressure (starting at about ≤500 mbar) and a temperature of 30° C. until reaching a final vacuum of ≤15 mbar.

### Example 2—Dissolution Behaviour of LASAG According to the Invention (Manually)

The dissolution behaviour of LASAG according to the second aspect of the invention (‘LASAG 2’) was tested in comparison to the prior art LASAG particles such as present, for instance, in Aspirin® i.v. For this purpose, and in order to be comparable to the commercially available Aspirin® i.v. vial, an 8 mL glass vial was filled with 1000 mg LASAG 2 particles and sealed with a stopper. An amount of 5 mL water for injection purposes was injected through the sealing stopper over the course of about 25-30 seconds, the needle then removed, and the vial shaken, or swirled, manually until complete dissolution (no residual solids visible to the human eye). The same test was performed with a commercially available Aspirin® i.v. vial. Each test was repeated at least 3 times per type of LASAG. This test and the resulting dissolution behaviour are considered similar to what is likely to be observed by, for instance, paramedics in an ambulance or medical staff in hospitals upon dissolving the dry LASAG particles in pre-filled vials by adding water for injection purposes and shaking manually. A more standardized dissolution test is provided in Example 3.

It was found that the ‘LASAG 2’ particles dissolved quicker and more readily than the prior art LASAG particles as present in Aspirin® i.v., with the ‘LASAG 2’ samples requiring only about 15-30 seconds and only 3-5 shaking/swirling motions from removing the needle until complete dissolution, versus about 40-60 seconds and using 8-10 shaking/swirling motions for the prior art LASAG particles as present in the Aspirin® i.v. samples.

### Example 3—Dissolution Behaviour of LASAG According to the Invention (Shaker)

The dissolution behaviour of LASAG according to the second aspect of the invention (‘LASAG 2’) was tested in a horizontal shaker (IKA®—Werke Gmbh & Co. KG) in comparison to the prior art LASAG particles such as present, for instance, in Aspirin® i.v. For this purpose, a 25 mL glass test tube was filled with 1000 mg LASAG 2 particles, and then placed onto the horizontal shaker. An amount of 5 mL water for injection purposes was added, and the vial shaken in intervals of 5 seconds at 1055 rpm until complete dissolution (no residual solids visible to the human eye). The same test was performed with a commercially available Aspirin® i.v. vial. Each test was repeated at least 3 times per type of LASAG.

Again, it was found that the ‘LASAG 2’ particles dissolved quicker and more readily than the prior art LASAG particles as present in Aspirin® i.v., with the ‘LASAG 2’ samples on average requiring only 18.3±2.9 seconds until complete dissolution, versus 23±2.9 seconds for the prior art LASAG particles as present in the Aspirin® i.v. samples. While this approximate 5 seconds difference might appear negligible, it should be understood that in emergency situations the less standardized manual dissolution procedure, as outlined in Example 2, is far more likely to occur than the use of a horizontal shaker.

### Example 4—Glass Adhesion Behaviour of LASAG According to the Invention

The glass adhesion behaviour of LASAG according to the second aspect of the invention (‘LASAG 2’) was tested in comparison to the prior art LASAG particles such as present, for instance, in Aspirin® i.v. For this purpose, and in order to be comparable to the commercially available Aspirin® i.v. vial, an 8 mL glass vial with an inner glass surface of approx. 29 cm² (estimated assuming the vial to be cylindrical) was filled with 1000 mg LASAG 2 particles and sealed with a stopper to prevent moisture absorption and/or pollutants falling into the vial during the test. On at least five consecutive days, the vial was manually moved, or rotated, once daily around all its axes to properly disperse the LASAG particles and allow them to coat the inner glass surface. After seven days, the vials were emptied by simply turning them upside down and allowing all non-adhering powder to spill out, or fall out, under gravity, followed by firmly tapping the vial five times onto a solid surface. No noteworthy adhesion to the stopper was observed. The weight of the remainder, i.e., the weight of the LASAG powder still sticking to the glass wall inside the vial after emptying, was determined gravimetrically. The same test was performed with a commercially available Aspirin® i.v. vial. Each test was repeated at least 3 times per type of LASAG.

It was found that the LASAG 2 particles adhered less to the inside of the glass vial than the Aspirin® i.v., with only 5.12±0.35 mg LASAG 2 remaining in the vial versus 9.00±1.58 mg of Aspirin® i.v. (equals about 0.177 mg/cm² versus about 0.312 mg/cm²). Moreover, there was also less fluctuation in the amount of LASAG 2 particles adhering to the glass than with Aspirin® i.v.

Reduced glass adhesion is considered beneficial insofar as drug that adheres to the glass at the top, e.g., near to the stopper, may be missed upon injection of the reconstitution medium (e.g., water for injection purposes) and could lead to dosing inaccuracies if the vial with the reconstituted LASAG-solution is not shaken, or not shaken often enough to retrieve and dissolve the adhering LASAG particles. Moreover, reduced glass adhesion also offers benefits during packaging procedures, especially at industrial scale, where the LASAG 2-particles allow for less sticking within the filling station.

### Example 5—Stability of LASAG According to the Invention in Solution

Due to its instability in water, LASAG is commonly used as a dry powder, and specifically as a dry powder for reconstitution; in other words, an aqueous solution thereof is not necessarily meant to be stored for extended periods of time but instead is prepared freshly prior to each use. Stability in aqueous solution is nonetheless an important feature for users, for instance, in order to know how long a prepared solution can still be used; a content of ≤1.5 wt.-% of the LASAG-degradant salicylic acid (SA) in a solution is typically considered acceptable. Therefore, the stability of LASAG according to the second aspect of the invention (‘LASAG 2’) in aqueous solution was tested in comparison to the prior art LASAG particles such as present, for instance, in Aspirin® i.v. For this purpose, an amount 1.0 g LASAG was dissolved in 5.0 mL water for injection purposes in crimped vials, and the respective contents of LASAG and the LASAG-degradant salicylic acid (SA) in solutions stored at room temperature (20±5° C.) and in cold storage (5±3° C.) were measured in sampled aliquots of 500 μL via HPLC (column) over time. In between sample time points, the vials were kept closed.

The following time points were measured:

| | | | | | | | | | | | | | |
|---|---|---|---|---|---|---|---|---|---|---|---|---|---|
| 20 ± 5° C. | t₀ | 0:40 | h | 01:20 | h | 2 h | 02:40 | h | 03:20 | h | 4 h | 6 h | 8 h |
| 5 ± 3° C. | t₀ | 1 | h | 2 | h | 4 h | 8 | h | 24 | h | 48 h | — | — |

None of the tested solutions (i.e., neither the ones with ‘LASAG 2’ nor the comparative ones with Aspirin® i.v.) exhibited changes in colour or clarity over the time range tested, or with the storage conditions tested.

For the HPLC-analysis, an Agilent Zorbax SB-C18 column was used (4.6×250 mm, 5 μm) at 22° C. with isocratic elution over 40 minutes at an eluent flow rate of 1 mL/min. The eluent was an 80 mM ammonium acetate/acetonitrile solution (60:40) prepared by dissolving 4 g ammonium acetate in 600 mL HPLC-grade water, adjusting the pH to 2.0 using trifluoroacetic acid, then adding 400 mL acetonitrile and homogenising the mixture. The injected sample volume was 10 μL. A UV-detector was used at 237 nm to analyse the samples.

The results are depicted in FIG. 2 which shows the increase in salicylic acid (SA) content (in weight percent) in aqeuous LASAG-solutions over time. As can be seen, when stored at room temperature, the ‘LASAG 2’-solutions exhibit the same stability as those of Aspirin® i.v., with both solutions exceeding 1.5 wt.-% SA content at about 1 h. Similar results were found with the solutions stored cold in the fridge at 5±3° C., with both solutions exceeding 1.5 wt.-% SA content at about 8 h (08:14 h for ‘LASAG 2’ vs. 07:59 h for Aspirin® i.v.). The latter result also shows that—while not preferred or recommended-reconstituted solutions can be used up to 8 h if stored continuously in a fridge at 5±3° C. right after reconstitution.

TABLE 1
Particle size distribution (PSD) data of different LASAG
grades (as measured by laser-diffraction):

| Particle size | Dᵥ10 | Dᵥ50 | Dᵥ90 | 1^{st} max. | 2^{nd} max. |
|---|---|---|---|---|---|
| distribution (PSD) | [μm] | [μm] | [μm] | [μm] | [μm] |
| LASAG 2⁺ | 4.2 | 18.4 | 53.3 | 20.4 | — |
| (mean of n = 4) | | | | | |
| LASAG 2⁺ | 3.8 | 16.3 | 40.4 | 18.7 | — |
| (batch in clinical study) | | | | | |
| Aspirin ® i.v.⁺⁺ (Bayer) | 6.9 | 34.2 | 200.0 | 35.3 | 211.0 |
| (n = 1) | | | | | |
| LASAG 1⁺⁺⁺ | 3.9 | 25.2 | 99.0 | 19.5 | 73.0 |
| (mean of n = 3) | | | | | |
| Aspegic ®⁺⁺⁺⁺ (Sanofi) | 12.5 | 64.1 | 177.0 | 98.1 | — |
| (n = 1) | | | | | |

⁺LASAG with ‘inner-crystalline’ glycine according to present invention (‘LASAG 2’)
⁺⁺Prior art LASAG with ‘inner-crystalline’ glycine from Bayer (presumably prepared as described in WO2006128600A1)
⁺⁺⁺Prior art LASA with ‘externally’ added glycine (‘LASAG 1’; preparation described in WO2018115434A1)
⁺⁺⁺⁺Prior art LASA, optionally with ‘externally’ added glycine from Sanofi

TABLE 2
Storage stability of LASAG according to present invention
in dry state, stored in aluminium-lined polyethylene-
bags under different storage conditions (n = 3)

| | | 25 ± 2° C. | 30 ± 2° C. | 40 ± 2° C. |
|---|---|---|---|---|
| | 5 ± 3° C. | 60 ± 5% r.h. | 65 ± 5% r.h. | 75 ± 5% r.h. |
| Time [months] | 70.9 ± 3.5 | 10.3 ± 2.0 | 3.5 ± 1.2 | 0.2 ± 0.0 |
| until reaching | | | | |
| salicylic acid | | | | |
| threshold of 1.5% | | | | |

The following list of numbered items are embodiments comprised by the present invention:

1. A method for the preparation of lysine acetylsalicylate⋅glycine (LASAG) comprising the steps of:
a) providing a solution of acetylsalicylic acid (ASA) in ethanol, said solution optionally being filtered in a step a₁;
b) adding glycine to the solution of step a to form a suspension;
c) providing an aqueous solution of lysine;
d) combining the solution of step c and the suspension of step b;
e) optionally stirring the suspension of step d;
f) adding acetone to the suspension of steps d or e;
g) incubating the suspension, optionally under stirring, to allow the formation of the lysine acetylsalicylate⋅glycine (LASAG) product;
h) isolating the lysine acetylsalicylate⋅glycine (LASAG) product of step g.

2. The method according to item 1, wherein the lysine acetylsalicylate⋅glycine (LASAG) is obtained in the form of a co-crystal.

3. The method according to any one of the preceding items, wherein acetylsalicylic acid is used in excess compared to lysine.

4. The method according to any one of the preceding items, wherein no seed crystals are added during the preparation; in particular, no seed crystals comprising, or consisting of, acetylsalicylic acid (ASA), lysine acetylsalicylate (LASA), or lysine acetylsalicylate⋅glycine (LASAG).

5. The method according to any one of the preceding items, wherein acetylsalicylic acid is used in excess compared to lysine, and no seed crystals are added during the preparation; in particular, no seed crystals comprising, or consisting of, acetylsalicylic acid (ASA), lysine acetylsalicylate (LASA) or lysine acetylsalicylate⋅glycine (LASAG).

6. The method according to any one of the preceding items, wherein the ethanolic ASA-solution of step a is prepared freshly prior to the subsequent steps.

7. The method according to any one of the preceding items, wherein the ethanolic ASA-solution of step a is prepared at elevated product temperatures, such as about 30±5° C., optionally in a reaction vessel equipped with a temperature-control jacket and at a jacket-temperature of about 30° C.

8. The method according to any one of the preceding items, wherein the ethanolic ASA-solution of step a is filtered in a step a₁, optionally sterile-filtered, prior to the subsequent steps.

9. The method according to any one of the preceding items, wherein the ethanolic ASA-solution of steps a or a₁ is cooled down to room temperature (20±5° C.) prior to step b, optionally under stirring to aid cool-down.

10. The method according to any one of the preceding items, wherein the glycine is added to the ethanolic ASA-solution of steps a or a₁ as a dry powder, optionally at a weight ratio of ASA to glycine in the range of 1:0.28-0.09, or 1:0.24-0.14.

11. The method according to any one of the preceding items, wherein the combination step d is performed by adding the lysine-solution of step c to the ASA-glycine-suspension of step b.

12. The method according to any one of the preceding items, wherein acetylsalicylic acid and lysine are used at a molar ratio in the range of 1:0.99-0.70, or 1:0.98-0.80, or 1:0.97-0.90, or 1:0.96-0.92; e.g., at a molar ratio of 1:0.9, or at a molar ratio of 1:0.95.

13. The method according to any one of the preceding items, wherein the ethanolic solution of acetylsalicylic acid provided in step a comprises, or consists of, about 8 to 20 wt.-%, or about 12 to 19 wt.-%, or about 14 to 18 wt.-% acetylsalicylic acid, and ethanol; e.g., about 15.7 wt.-%.

14. The method according to any one of the preceding items, wherein the aqueous solution of lysine provided in step c comprises, or consists of, about 41 to 55 wt.-% lysine, and water.

15. The method according to any one of the preceding items, wherein the aqueous solution of lysine provided in step c was prepared from lysine monohydrate, optionally L- or D,L-lysine monohydrate.

16. The method according to any one of the preceding items, wherein the volume of the ethanolic ASA-solution in step a exceeds the volume of the acetone added in step f.

17. The method according to any one of the preceding items, wherein at least method steps b to g are performed at room temperature (20±5° C.), or below.

18. The method according to any one of the preceding items, wherein at least the incubation step g is performed at room temperature (20±5° C.).

19. The method according to any one of the preceding items, wherein the product isolation step h is performed by filtration.

20. The method according to any one of the preceding items, wherein the product isolation step h is performed by centrifugation.

21. The method according to any one of the preceding items, wherein the product isolation step h is performed by filtration and centrifugation.

22. The method according to any one of the preceding items, additionally comprising a step i of washing the isolated product.

23. The method according to item 22, wherein the washing step i involves washing the isolated product with ethanol and/or acetone.

24. The method according to items 22 to 23, wherein the washing step i involves washing the isolated product with ethanol, preferably washing it with ethanol twice.

25. The method according to items 22 to 24, wherein the washing step i involves washing the isolated product with ethanol, followed by washing the isolated product with acetone; optionally repeating these washing steps.

26. The method according to any one of the preceding items, wherein the isolated and washed product is dried under reduced pressure, optionally at a temperature of about 30° C.

27. The method according to any one of the preceding items, wherein the method is performed under sterile conditions.

28. The method according to any one of items 1 to 27, wherein the method is performed under non-sterile conditions and comprises an additional step of sterilizing the product obtained in steps h or i by irradiation, optionally gamma-irradiation.

29. The method according to any one of the preceding items, wherein the particles of the product obtained in steps h or i have a median particle size (D50) of less than 40 μm, or less than 30 μm, or less than 20 μm.

30. The method according to any one of the preceding items, wherein at least 90% of the particles obtained in steps h or i have a particle size (D90) of less than 65 μm, or less than 55 μm, or less than 45 μm.

31. The method according to any one of the preceding items, wherein the particles of the product obtained in steps h or i have a median particle size (D50) of less than 40 μm, and at least 90% of the particles have a particle size (D90) of less than 65 μm; or
wherein the particles of the product obtained in steps h or i have a median particle size (D50) of less than 30 μm, and at least 90% of the particles have a particle size (D90) of less than 55 μm; or
wherein the particles of the product obtained in steps h or i have a median particle size (D50) of less than 20 μm, and at least 90% of the particles have a particle size (D90) of less than 45 μm.

32. The method according to any one of the preceding items, wherein the particles of the product obtained in steps h or i exhibit a D90/D50 ratio of 3.7 or less, or 3.4 or less, or 3.1 or less.

33. The method according to any one of the preceding items, wherein the particles of the product obtained in steps h or i exhibit a unimodal particle size distribution.

34. The method according to any one of the preceding items, wherein the method exhibits a yield of at least 90%, or at least 92%, or at least 94%, or at least 96%.

35. The method according to any one of the preceding items, wherein the method exhibits a yield in the range of 90% to 100%, or 94% to 99%, or 96% to 98%.

36. The method according to any one of the preceding items, wherein the aqueous solution of lysine provided in step c further comprises dissolved glycine.

37. A lysine acetylsalicylate⋅glycine (LASAG) obtainable by, or obtained by, a method according to any one of the preceding claims.

38. The lysine acetylsalicylate⋅glycine according to item 37, wherein the lysine acetylsalicylate⋅glycine (LASAG) is in the form of a co-crystal.

39. The lysine acetylsalicylate⋅glycine according to items 37 to 38, wherein the particles have a median particle size (D50) of less than 40 μm, or less than 30 μm, or less than 20 μm.

40. The lysine acetylsalicylate⋅glycine according to items 37 to 39, wherein at least 90% of the particles have a particle size (D90) of less than 65 μm, or less than 55 μm, or less than 45 μm.

41. The lysine acetylsalicylate⋅glycine according to items 37 to 40, wherein the particles have a median particle size (D50) of less than 40 μm, and at least 90% of the particles have a particle size (D90) of less than 65 μm;
wherein the particles have a median particle size (D50) of less than 30 μm, and at least 90% of the particles have a particle size (D90) of less than 55 μm; or
wherein the particles have a median particle size (D50) of less than 20 μm, and at least 90% of the particles have a particle size (D90) of less than 45 μm.

42. The lysine acetylsalicylate⋅glycine according to items 37 to 41, wherein the particles exhibit a D90/D50 ratio of 3.7 or less, or 3.4 or less, or 3.1 or less.

43. The lysine acetylsalicylate⋅glycine according to items 37 to 42, wherein the particles exhibit a unimodal particle size distribution.

44. The lysine acetylsalicylate⋅glycine according to items 37 to 43, wherein the particles exhibit low adhesion to glass surfaces.

45. The lysine acetylsalicylate⋅glycine according to items 37 to 44, wherein the particles exhibit a specific surface area, as measured via BET gas adsorption technique, which is 1.50 m²/g or higher, or 1.75 m²/g or higher, or 2.00 m²/g or higher, or 2.25 m²/g or higher.

46. The lysine acetylsalicylate⋅glycine according to items 37 to 45, wherein the particles exhibit a density of 1.450 g/cm³ or higher, or 1.460 g/cm³ or higher, or 1.470 g/cm³ or higher.

47. A lysine acetylsalicylate⋅glycine according to items 37 to 46, for use as a medicine.

48. A lysine acetylsalicylate⋅glycine according to items 37 to 46, for use in:
the treatment and/or prevention of viral infections in humans or animals;
the treatment and/or prevention of acute coronary syndromes (including instable angina and myocardial infarction);
the treatment of fever;
the treatment of acute moderate to strong pains (including migraine headaches).

1. A method for the preparation of lysine acetylsalicylate⋅glycine (LASAG) comprising the steps of:
a) providing a solution of acetylsalicylic acid (ASA) in ethanol, said solution optionally being filtered in a step a₁;
b) adding glycine to the solution of step a to form a suspension;
c) providing an aqueous solution of lysine;
d) combining the solution of step c and the suspension of step b;
e) optionally stirring the suspension of step d;
f) adding acetone to the suspension of steps d or e;
g) incubating the suspension, optionally under stirring, to allow the formation of the lysine acetylsalicylate⋅glycine (LASAG) product;
h) isolating the lysine acetylsalicylate⋅glycine (LASAG) product of step g.

2. The method according to claim 1, wherein the lysine acetylsalicylate⋅glycine (LASAG) is obtained in the form of a co-crystal.

3. The method according to any one of the preceding claims, wherein acetylsalicylic acid is used in excess compared to lysine; and/or
wherein no seed crystals are added during the preparation.

4. The method according to any one of the preceding claims, wherein the glycine is added to the ethanolic ASA-solution of steps a or a₁ as a dry powder.

5. The method according to any one of the preceding claims, wherein the ethanolic solution of acetylsalicylic acid provided in step a comprises, or consists of, about 8 to 20 wt.-%, or about 12 to 19 wt.-%, or about 14 to 18 wt.-% acetylsalicylic acid, and ethanol; and/or
wherein the aqueous solution of lysine provided in step c comprises, or consists of, about 41 to 55 wt.-% lysine, and water.

6. The method according to any one of the preceding claims, wherein at least method steps b to g are performed at room temperature (20±5° C.), or below.

7. The method according to any one of the preceding claims, wherein the product isolation step h is performed by filtration and/or centrifugation.

8. The method according to any one of the preceding claims, additionally comprising a step i of washing the isolated product; optionally washing the isolated product with ethanol and/or acetone.

9. The method according to any one of the preceding claims, wherein the method exhibits a yield of at least 90%, or at least 92%, or at least 94%, or at least 96%.

10. A lysine acetylsalicylate⋅glycine (LASAG) obtainable by, or obtained by, a method according to any one of the preceding claims.

11. The lysine acetylsalicylate⋅glycine according to claim 10, wherein the lysine acetylsalicylate⋅glycine (LASAG) is in the form of a co-crystal.

12. The lysine acetylsalicylate⋅glycine according to any one of claims 10 to 11, wherein the particles have a median particle size (D50) of less than 40 μm, or less than 30 μm, or less than 20 μm; and/or
wherein at least 90% of the particles have a particle size (D90) of less than 65 μm, or less than 55 μm, or less than 45 μm.

13. The lysine acetylsalicylate⋅glycine according to any one of claims 10 to 12, wherein the particles exhibit a unimodal particle size distribution and/or low adhesion to glass surfaces.

14. A lysine acetylsalicylate⋅glycine according to any one of claims 10 to 13, for use as a medicine.

15. A lysine acetylsalicylate⋅glycine according to any one of claims 10 to 13, for use in the treatment and/or prevention of viral infections in humans or animals.
